(19) United States
(12) Patent Application Publication
(10) Pub. No.: US 2024/0389968 A1
(43) Pub. Date: Nov. 28, 2024

(54) CAPABILITY DETERMINATION METHOD, CAPABILITY DETERMINATION APPARATUS, CAPABILITY DETERMINATION PROGRAM, AND PHANTOM

(71) Applicant: Konica Minolta, Inc., Tokyo (JP)

(72) Inventor: Tatsuya TAKAGI, Tokyo (JP)

(21) Appl. No.: 18/671,271

(22) Filed: May 22, 2024

(30) Foreign Application Priority Data
May 24, 2023 (JP) 2023-085708

(51) Int. Cl. A61B 6/58; A61B 6/50

(52) U.S. Cl. CPC A61B 6/585; A61B 6/503; A61B 6/504; A61B 6/583

(57) ABSTRACT

A capability determination method includes: acquiring an imaging capability from an image obtained by a radiation detector by irradiation of a model with radiation; and outputting the acquired imaging capability in such a manner as to indicate a relation with a required imaging capability for a predetermined dynamic analysis.

### CROSS REFERENCE TO RELATED APPLICATIONS

The entire disclosure of Japanese Patent Application No. 2023-085708 filed on May 24, 2023 is incorporated herein by reference in its entirety.

### BACKGROUND

### Technological Field

The present disclosure relates to a capability determination method, a capability determination apparatus, a capability determination program, and a phantom.

### Description of Related Art

In recent years, there has been proposed a radiography system that generates a dynamic radiographic image (hereinafter, simply referred to as a “dynamic image”) from a plurality of radiographic still images (hereinafter, simply referred to as “still images”) obtained by sequentially imaging an imaging target with irradiation (for example, Japanese Unexamined Patent Publication No. 2022-11259). Since the dynamic image can visualize various motions in a living body, the dynamic image can be used for clinical diagnosis of various pathological conditions that cannot be diagnosed with a conventional still image. A radiographic system for generating the dynamic image is capable of improving diagnostic accuracy by performing dynamic analysis and dynamic image processing corresponding to a diagnostic purpose on a plurality of still images and displaying a dynamic image after the analysis and image processing.

For example, Patent Literature 1 describes a quality control method for efficiently performing quality control of dynamic imaging by outputting information about the quality of a dynamic image obtained by imaging for generating the dynamic image (hereinafter referred to as “dynamic imaging”) and information about the function of a radiography apparatus used for the dynamic imaging.

### SUMMARY

However, in a case where an image obtained by its radiation detector is used for various diagnoses, only reference to information regarding the function does not make it possible to accurately confirm in advance whether or not the radiation detector has a performance (imaging capability) suitable for a diagnosis purpose, whether or not an image resolution corresponding to an actual diagnosis site can be obtained, and the like.

In order to stop unnecessary imaging and reduce the number of times of exposure of a patient, an apparatus to be used surely needs to have performance suitable for a diagnostic purpose, but there has been no proposal to confirm, before diagnosis, the imaging capability of a radiation detector or a radiography apparatus according to the diagnostic purpose.

An object of the present disclosure is to provide a capability determination method, a capability determination apparatus, a capability determination program, and a phantom, making it possible to perform quality control of dynamic imaging in association with a diagnostic purpose.

A capability determination method performed by a dynamic quality control apparatus according to an embodiment of the present disclosure includes: acquiring an imaging capability from an image obtained by a radiation detector by irradiation of a model with radiation; and outputting the acquired imaging capability in such a manner as to indicate a relation with a required imaging capability for a predetermined dynamic analysis.

A capability determination apparatus according to an embodiment of the present disclosure includes: an acquirer that acquires an imaging capability from an image obtained by a radiation detector by irradiation of a model with radiation; and an output that outputs the acquired imaging capability in such a manner as to indicate a relation with a required imaging capability for a predetermined dynamic analysis.

A capability determination program according to an embodiment of the present disclosure is stored in a computer-readable recording medium and causes a computer to execute the above-described capability determination method.

A phantom according to an embodiment of the present disclosure is a phantom used as a model in the above-described capability determination method, and the phantom includes a plurality of regions having different radiolucencies and a plurality of granules having different sizes, the plurality of granules being disposed at positions downstream of the plurality of regions along passing radiation.

A determination method for determining a dynamic imaging capability of a radiography apparatus according to an embodiment of the present disclosure is a determination method for determining a dynamic imaging capability of a radiography apparatus and includes: preparing a model whose radiolucency and movement speed are changeable; acquiring a plurality of images continuously captured by irradiation of the model with radiation, while selecting a combination of the radiolucency and the movement speed of the model; comparing a resolution of each the plurality of image acquired and a reference value set for each imaging purpose; and determining, based on a result of the comparison, whether or not the radiography apparatus has a required imaging capability.

A determination apparatus for determining a dynamic imaging capability of a radiography apparatus according to an embodiment of the present disclosure is a determination apparatus for determining a dynamic imaging capability of a radiography apparatus and includes: an image acquirer that acquires a plurality of images continuously captured by irradiation of the model with radiation, while selecting a combination of a radiolucency and a movement speed of a model whose radiolucency and movement speed are changeable; a comparison section that compares a resolution of each the plurality of image acquired and a reference value set for each imaging purpose; and a determiner that determines, based on a result of the comparison, whether or not the radiography apparatus has a required imaging capability.

Note that these generic or specific aspects may be implemented as a system, an apparatus, a method, an integrated circuit, a computer program, or a recording medium, or any selective combination of a system, an apparatus, a method, an integrated circuit, a computer program, and a recording medium.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention:

FIG. 1 is a diagram illustrating a configuration of a radiography system;

FIG. 2 is a block diagram illustrating a common configuration among a console, an analysis apparatus, and a dynamic quality control apparatus;

FIG. 3 is a diagram illustrating a capability determination process performed by the dynamic quality control apparatus;

FIG. 4 is a diagram illustrating a configuration of a phantom;

FIG. 5 is a plan view of the phantom;

FIG. 6 is a diagram illustrating details of determination as to whether or not a still image satisfies reference quality;

FIG. 7 is a diagram illustrating a determination result as to whether or not the information regarding a contrast resolution and a time resolution for each body thickness at which the phantom is imaged by a radiography apparatus satisfies the quality;

FIG. 8 is a diagram illustrating an example of reference quality information required for generating a dynamic image in each evaluation mode; and

FIG. 9 is a diagram illustrating a determination result as to whether or not the radiography apparatus has the capabilities of the body thickness, the contrast resolution, and the time resolution necessary in each evaluation mode.

### DETAILED DESCRIPTION OF EMBODIMENTS

Hereinafter, one or more embodiments of the present invention will be described with reference to the drawings. However, the scope of the invention is not limited to the disclosed embodiments. In the following, embodiments of the present disclosure will be described in detail with reference to the drawings as appropriate.

### 1. Radiography System

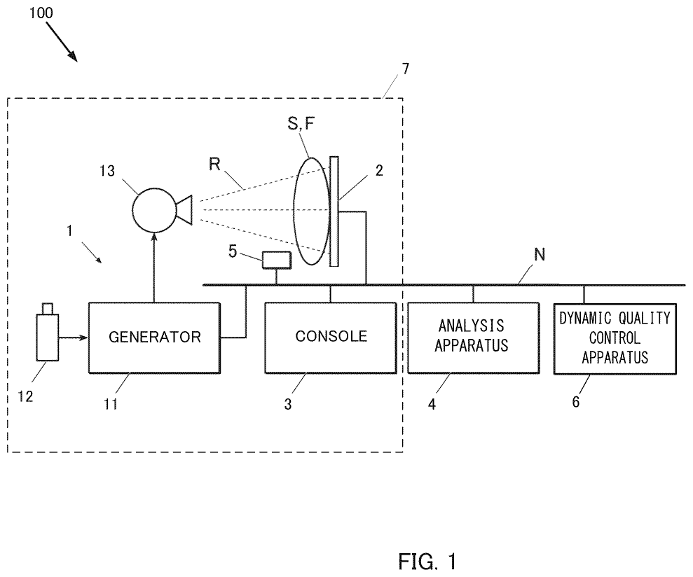

First, a schematic configuration of a radiography system 100 according to an embodiment of the present disclosure will be described. FIG. 1 is a view illustrating a configuration of the radiography system 100.

The radiography system 100 includes a radiation generating apparatus 1, a radiation detector 2, a console 3, an analysis apparatus 4, an optical camera 5, and a dynamic quality control apparatus 6. The radiation generating apparatus 1, the radiation detector 2, the console 3, the analysis apparatus 4, the optical camera 5, and the dynamic quality control apparatus 6 can be connected by a network N to communicate with each other. The radiation generating apparatus 1 and the radiation detector 2 constitute a radiography apparatus 7. The console 3 may be included in the radiography apparatus 7. FIG. 1 illustrates a case where the console 3 is included in the radiography apparatus 7. The radiography apparatus 7 is a radiography apparatus operable to perform dynamic imaging

Note that the radiography system 100 may be communicable with a Hospital Information System (HIS), a Radiology Information System (RIS), a Picture Archiving and Communication System (PACS), a dynamic analysis apparatus, and the like.

The network N connects the radiation generating apparatus 1, the radiation detector 2, the console 3, the analysis apparatus 4, the optical camera 5, and the dynamic quality control apparatus 6. The network N may be a local area network (LAN), a wide area network (WAN), the Internet, a public line, a dedicated line, or the like, and may be a wireless line or a wired line, or a mixture of the wireless line and the wired line.

Hereinafter, the radiation generating apparatus 1, the radiation detector 2, the console 3, the analysis apparatus 4, the optical camera 5, and the dynamic quality control apparatus 6 will be described in order.

### [1-1. Radiation Generating Apparatus]

The radiation generating apparatus 1 includes a generator 11, an irradiation instruction switch 12, and a radiation source 13. The radiation generating apparatus 1 may be installed in an imaging room, or may be movable together with the radiation detector 2, the console 3, and the like, for example, in a vehicle called a medical cart.

The generator 11 applies, to the radiation source 13 (tube bulb), a voltage corresponding to imaging conditions preset by the console 3, based on operation of the irradiation instruction switch 12. The imaging conditions are, for example, conditions related to a subject S, such as an imaging portion, an imaging direction, and a physique, and conditions related to irradiation with radiation R, such as a tube voltage, a tube current, an irradiation time, a current-time product (mAs value), and a pulse cycle, and are set in advance by the console 3.

The irradiation instruction switch 12 is a switch for controlling whether the generator 11 applies a voltage to the radiation source 13.

The radiation source 13 is a tube bulb and emits, from an irradiation port, the radiation R of a dose corresponding to the voltage applied from the generator 11. The radiation R is, for example, X-rays. The radiation source 13 is movable in an X-axis direction, a Y-axis direction, and a Z-axis direction that are orthogonal to each other, and is rotatable about a rotation axis parallel to the Y-axis and/or the Z-axis to change the direction of the irradiation port of the radiation.

The radiation generating apparatus 1 generates the radiation R after the imaging conditions are set by the console 3 and when a voltage corresponding to the set imaging conditions is applied to the radiation source 13 under the control of the irradiation instruction switch 12. In a case in which the dynamic image is captured, the imaging conditions are set by the console 3 such that the irradiation with the pulsed radiation R is repeated a plurality of times per predetermined time (for example, 15 times per second) for each depression of the irradiation instruction switch 12. In a case where the pulsed radiation R cannot be repeated a plurality of times per predetermined time due to the capability of the radiation generating apparatus 1, the irradiation with the radiation R may be continued for a predetermined time.

That is, “irradiating with radiation for a predetermined time” in the present disclosure includes both pulse irradiation in which irradiation with the pulsed radiation R is repeated a plurality of times over a predetermined time and continuous irradiation in which irradiation with the radiation R is continued for a predetermined time. The time for irradiation with the radiation R is, for example, 15 seconds. In a case in which the pulse irradiation is performed for capturing the dynamic image, it is possible to set the irradiation dose for irradiation of the patient as a whole to the same amount as in a case in which the still image of the chest is captured, since the irradiation amount of the radiation R for irradiation by one pulse may be smaller than the irradiation amount of the radiation in a case in which the still image is captured. Note that in the case of continuous irradiation with the radiation R, it is desirable to set imaging conditions that do not significantly increase the amount of exposure of the patient.

### [1-2. Radiation Detector]

Although not illustrated, the radiation detector 2 includes a sensor substrate, a scanning circuit, a readout circuit, a controller, a communicator, and the like. In the sensor substrate, pixels are arranged two dimensionally (in a matrix). Each pixel includes a radiation detection element that generates charges corresponding to the dose of the radiation R received through the subject S, and a switch element that accumulates and discharges the charges. The scanning circuit switches each switch element on or off. The readout circuit reads out the amount of charge emitted from each pixel as a signal value (intensity). The controller generates a radiographic image from a plurality of signal values read by the reading circuit. The communicator transmits data of the radiographic image generated by the readout circuit, various signals, and the like to the outside, and receives various kinds of information and various signals.

In each pixel of the radiation detector 2 configured in such a manner, when the radiation detection element receives the radiation R in a state in which the scanning circuit turns off the switch element, the radiation detection element generates and accumulates an electric charge corresponding to the dose of the radiation R. When the scanning circuit turns on the switch element, the accumulated charge is released, the readout circuit detects the amount of charges released from the respective pixels and generates signal values indicating the amount of charges, and the controller generates a radiographic image based on the signal values generated for the respective pixels. The generated individual radiographic images are one still images in the dynamic imaging. In the case of pulse irradiation, one still image is generated corresponding to each pulse.

In order to generate a dynamic image, the radiation detector 2 repeats the generation of one still image by the above-described series of operations at a cycle of a plurality of times per unit time (for example, 15 times per second) for each pressing of the irradiation instruction switch 12 over a predetermined time (duration). Here, the pulse period and duration are preset by the console 3. In the case of pulse irradiation, the generation of the plurality of still images constituting the dynamic image is synchronized with the timing at which the radiation R is emitted from the radiation generating apparatus 1. In the case of continuous irradiation, a plurality of still images constituting a dynamic image are generated during the time of continuous irradiation.

Note that the generated still image may be stored in the radiography system 100 as image data, may be transferred to the outside of the radiography system 100 (for example, a data storage apparatus such as a cloud server or a PACS), or may be displayed in real time inside or outside the radiography system 100.

### [1-3. Console]

The console 3 sets various imaging conditions (a tube voltage, a tube current, an irradiation time (mAs value), an imaging region, an imaging direction, and the like) in at least the radiation detector 2. In a case where the radiation generating apparatus 1 performs pulse irradiation, the console 3 also sets various imaging conditions for the radiation generating apparatus 1. The console 3 sets imaging conditions based on imaging order information acquired from another system (HIS, RIS, or the like) or an operation performed by a user (e.g., a technician).

The console 3 includes a personal computer (PC), a dedicated device, or the like. In the present embodiment, the console 3 includes a controller 201, a storage 202, a display 203, a communicator 204, and an operation unit 205 as a basic configuration. The basic configuration is as follows.

The controller 201 is formed with a hardware processor such as a central processing unit (CPU) or a digital signal processor (DSP).

The storage 202 includes a nonvolatile memory, a hard disk, or the like. The storage 202 may be built in the console 3 or may be detachably attached to the console 3. The storage 202 stores a program to be executed by the controller 201, parameters necessary for executing the program, data necessary for executing the program, and data obtained by executing the program. Note that the program may be stored in the storage 202 in advance, or May be downloaded from the outside via the communicator 204. The storage 202 is an example of a non-transitory computer-readable recording medium.

The display 203 displays various information such as an image. The display 203 includes, for example, a liquid crystal display (LCD), an electronic luminescent display (ELD), a cathode ray tube (CRT), or the like.

The communicator 204 includes a communication module and the like. The communicator 204 transmits and receives various signals and various data to and from an external device connected via the network N.

The operation unit 205 is an operation means configured to be operable by a user. The operation unit 205 includes a keyboard, a pointing device such as a mouse, and a touch panel laminated on a surface of the display 203. Next, the operation unit 205 outputs a signal corresponding to the operation performed by the user to the controller 201.

The above-described basic configuration is common to the console 3, the analysis apparatus 4, and the dynamic quality control apparatus 6.

The controller 201 of the console 3 reads various programs, parameters, and data for controlling the operation of the console 3 stored in the storage 202 of the console 3, executes various processes in accordance with the read programs, and controls the operations of the storage 202, the display 203, the communicator 204, and the operation unit 205 of the console 3.

### [1-4. Analysis Apparatus]

The analysis apparatus 4 is an apparatus that generates a dynamic image based on the radiographic image detected by the radiation detector 2. The analysis apparatus 4 includes a PC, a dedicated device, or the like. In the present embodiment, similarly to the console 3 and the dynamic quality control apparatus 6, the analysis apparatus 4 includes, as a basic configuration, a controller 201, a storage 202, a display 203, a communicator 204, and an operation unit 205.

The controller 201 of the analysis apparatus 4 reads various programs, parameters, and data for operation control of the analysis apparatus 4 stored in the storage 202 of the analysis apparatus 4, executes various processes in accordance with the read programs, and controls the operations of the storage 202, the display 203, the communicator 204, and the operation unit 205 of the analysis apparatus 4.

The various processes executed by the controller 201 of the analysis apparatus 4 include a dynamic analysis process including an evaluation mode. In the dynamic analysis process, the dynamic aspect of the subject S or the like (for example, the movement of an organ, the flow of a fluid, or the like) is analyzed based on the image captured by the radiography apparatus 7, and image processing reflecting the analysis result is performed on the dynamic image, thereby generating a dynamic image after the analysis. The dynamic image is generated in accordance with each mode processing such as a mode for performing blood flow deficit evaluation, a mode for performing pulmonary artery regurgitation ratio evaluation, and a mode for performing left ventricular ejection capability evaluation. The generated dynamic image is stored in the storage 202 of the analysis apparatus 4, and is displayed on the display 203 of the analysis apparatus 4 in response to a user operation or the like on the operation unit 205 of the analysis apparatus 4. The dynamic image is transferred to and stored in another apparatus in the radiography system 100 or the outside of the radiography system 100 via the communicator 204 of the analysis apparatus 4. The dynamic analysis process refers to a series of operations including generation of a dynamic image by dynamic analysis and image processing and storage of the generated dynamic image.

The analysis apparatus 4 may be integrated with the console 3, or may be integrated with the dynamic quality control apparatus 6.

The analysis apparatus 4 may not include the display 203 and the operation unit 205. For example, the analysis apparatus 4 may receive a control signal from the operation unit 205 provided separately from the analysis apparatus 4 via the communicator 204 or the like, and output an image signal such as a dynamic image to the display 203 provided separately from the analysis apparatus 4.

### [1-5. Optical Camera]

The optical camera 5 is a camera that optically images the phantom F when a dynamic image for performing quality control using the phantom F is obtained.

### [1-6. Dynamic Quality Control Apparatus]

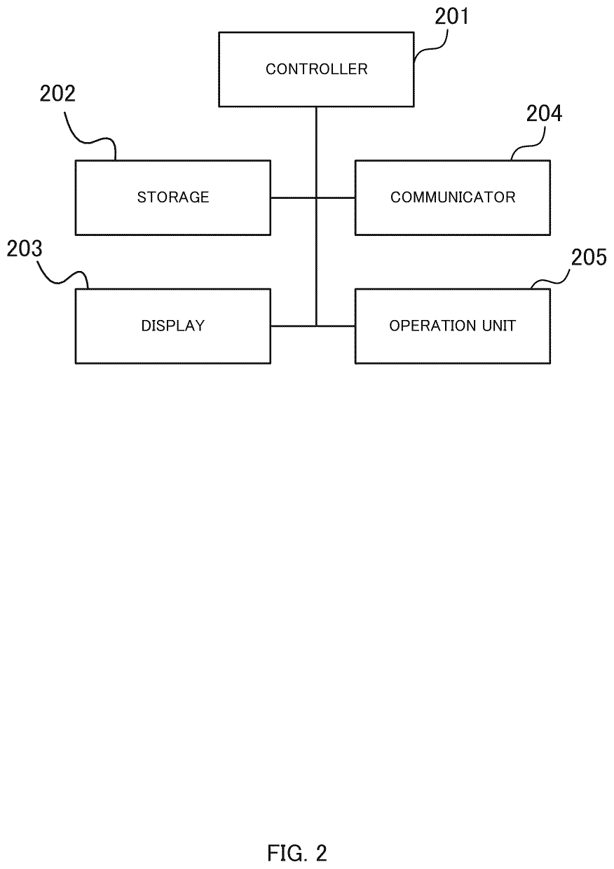

The dynamic quality control apparatus 6 is a device for determining the capability of dynamic imaging. FIG. 2 is a diagram illustrating a schematic configuration of the dynamic quality control apparatus 6.

The dynamic quality control apparatus 6 includes a PC, a dedicated device, or the like. In the present embodiment, similarly to the console 3 and the analysis apparatus 4, the dynamic quality control apparatus 6 includes, as basic components, a controller 201, a storage 202, a display 203, a communicator 204, and an operation unit 205.

The dynamic quality control apparatus 6 may not include the display 203 and the operation unit 205, may receive a control signal from an input device provided separately from the dynamic quality control apparatus 6, for example, the operation unit 205 of the console 3 via the communicator 204 or the like, and may output an image signal to a display device provided separately from the dynamic quality control apparatus 6, for example, the display 203 of the console 3.

The controller 201 of the dynamic quality control apparatus 6 reads various programs, parameters, and data for controlling the operation of the dynamic quality control apparatus 6 stored in the storage 202 of the dynamic quality control apparatus 6, executes various processes according to the read programs, and controls the operations of the storage 202, the display 203, the communicator 204, and the operation unit 205 of the dynamic quality control apparatus 6. The programs stored in the storage 202 of the dynamic quality control apparatus 6 include a program for causing the controller 201 of the dynamic quality control apparatus 6 to execute dynamic quality control processing (an example of a quality control method for dynamic imaging) described in detail below.

Here, as a quality control method of dynamic imaging, there is the above-described quality control method in the related art. However, as described above, there has been no proposal to confirm the performance of a radiography apparatus according to a diagnostic purpose before diagnosis.

Even if the user views a plurality of still images generated by the radiography apparatus in order to evaluate whether the performance of the radiography apparatus is suitable for the purpose of diagnosis, it is difficult to determine whether the still images have the quality required to generate a dynamic image corresponding to a pathological condition. In addition, when the user views the dynamic image after the image processing, it is also difficult to determine whether or not the still image before the image processing has necessary quality, that is, whether or not the dynamic image after the image processing is an accurate dynamic image.

In performing the dynamic analysis, if a still image which does not satisfy the quality required for the evaluation mode is used, as a result, accurate diagnosis becomes difficult, and in some cases, re-imaging may be required.

In order to perform accurate diagnosis, it is necessary to use a radiography apparatus having a predetermined capability. However, there are a wide variety of radiation detectors or radiography apparatuses installed in different hospitals. Therefore, in a case of determining whether or not a radiation detector or a radiography apparatus to be used has a capability necessary for the evaluation mode, that is, whether or not the radiation detector or the radiography apparatus has a capability to perform dynamic imaging corresponding to each evaluation mode, it is necessary to perform the determination individually for the radiation detector or the radiography apparatus to be actually used, which is laborious and difficult.

Furthermore, as the research and development of dynamic imaging have progressed, the present inventors have found that the quality required for the radiation detector or the radiography apparatus, more specifically, the resolution required for dynamic analysis process (required resolution) varies depending on the evaluation mode. For example, the required resolution is high in one evaluation mode corresponding to one diagnostic purpose, and the required resolution is low in another evaluation mode corresponding to another diagnostic purpose. Therefore, merely grasping the functions and specifications of the used apparatus and attribute data such as the frame rate of the captured image is not necessarily sufficient for determining whether the apparatus and the image are suitable for use in a specific evaluation mode. In view of this, the present inventors have conducted intensive studies on a quality control method that makes it possible to confirm the performance of a radiation detector or a radiography apparatus according to a diagnostic purpose before diagnosis, and as a result, have proposed a dynamic quality control process described in the present embodiment.

That is, the dynamic quality control apparatus 6 is an apparatus that determines whether the radiation detector 2 or the radiography apparatus 7 satisfies the capability required for each evaluation mode.

The dynamic quality control apparatus 6 determines whether a frame (still image) obtained by the radiation detector 2 when the radiography apparatus 7 images a model called a phantom F (an example of a quality control model for dynamic imaging) in place of a human body has quality necessary for each dynamic analysis process, thereby determining whether the radiation detector or the radiography apparatus 7 satisfies the imaging capability necessary for each dynamic analysis process.

In a medical institution including a plurality of radiation detectors 2 and one or more radiation generators 1, the radiation detector 2 used for the radiation generating apparatus 1 may be changed according to imaging. In such a case, the still image satisfying the required quality is influenced by the imaging capability of the radiation detector 2. Therefore, the dynamic quality control apparatus 6 can determine the imaging capability of the radiation detector 2.

The imaging capability may also be influenced by the combination of the radiation detector 2 and the radiation generating apparatus 1. In this case, the imaging capability is influenced by the imaging capability of the radiography apparatus 7 including the radiation detector 2 and the radiation generating apparatus 1, which have the capability to obtain a still image satisfying the required quality. Therefore, the dynamic quality control apparatus 6 can also determine the imaging capability of the radiography apparatus 7 including the radiation detector 2 and the radiation generating apparatus 1.

Furthermore, the captured image may be influenced by the control capability of the console 3 that controls the radiation detector 2 and the radiation generating apparatus 1. In this case, the captured image is influenced by the imaging capability of the radiography apparatus 7 including: the radiation detector 2 and the radiation generating apparatus 1 which have the capability to obtain a still image satisfying the required quality; and the console 3. Therefore, the dynamic quality control apparatus 6 can also determine the imaging capability of the radiography apparatus 7 including the radiation detector 2, the radiation generating apparatus 1, and the console 3.

The method or program of the present disclosure is executed by the dynamic quality control apparatus 6. Details will be described later.

The “quality control” includes quality assurance at the time of delivery of a product such as the radiation generating apparatus 1 or the radiation detector 2, in addition to quality control by periodic quality checks after the delivery of the product.

Note that the dynamic quality control apparatus 6 may be integrated with the console 3, may be integrated with the analysis apparatus 4, or may be integrated with both the console 3 and the analysis apparatus 4.

When the radiography system 100 includes a PACS (not illustrated) or the like, the dynamic quality control apparatus 6 may be integrated with these devices.

### 2. Details of Dynamic Quality Control Apparatus

### [2-1. Processing by Dynamic Quality Control Apparatus]

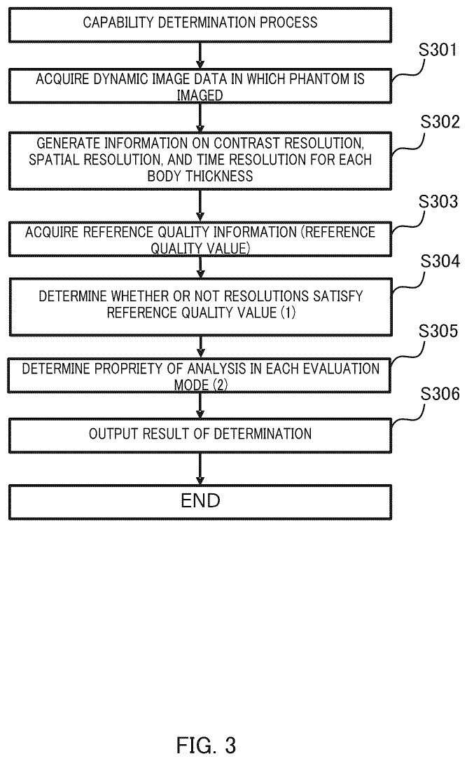

FIG. 3 is a flowchart illustrating an overview of a capability determination process performed by the controller 201 of the dynamic quality control apparatus 6. Hereinafter, the controller 201 of the dynamic quality control apparatus 6 is simply referred to as a “controller 201”.

First, the controller 201 acquires still images of the phantom F captured as the subject S by the radiation detector 2 (S301). In order to determine whether the radiation detector 2 or the radiography apparatus 7 satisfies the imaging capability necessary for each dynamic analysis process, it is necessary to obtain an image obtained by imaging an identical imaging target. Therefore, the radiography apparatus 7 does not image a human body but images a model used for radiation imaging, which is called a phantom F. When the radiography apparatus 7 images the phantom F, it is possible to acquire the resolution of the radiation detector 2 or the radiography apparatus 7, that is, the contrast resolution, the spatial resolution, and the time resolution of each body thickness. The imaging capability necessary for each dynamic analysis process is a required resolution, that is, a contrast resolution, a spatial resolution, and a time resolution for each body thickness. By setting a phantom F corresponding to a contrast resolution, a spatial resolution, and a time resolution for each body thickness for determination, and imaging the phantom F, it is possible to determine whether or not the captured still image has a specific contrast resolution, a spatial resolution, and a specific time resolution of each body thickness. The setting of the phantom F will be described later.

Next, the controller 201 generates, for the acquired still images, the resolutions, that is, information on the contrast resolution, the spatial resolution, and the time resolution for each body thickness (S302). The body thickness, the contrast resolution, the spatial resolution, and the time resolution generated in step S302 are equal to the body thickness, the contrast resolution, the spatial resolution, and the time resolution set for the phantom F when the still images are captured in step S301.

Then, the controller 201 acquires reference quality information required for each evaluation mode, which is necessary for evaluating the pathological condition (S303). The reference quality information is the required resolution in each evaluation mode and is information on the contrast resolution, the spatial resolution, and the time resolution for each body thickness required to generate a dynamic image.

Next, the controller 201 determines whether the still images captured with the body thickness, the contrast resolution, the spatial resolution, and the time resolution acquired and generated in steps S301 and S302 satisfy the reference qualities (S304). Details of the determination will be described later.

Then, the controller 201 determines whether the radiation detector 2 or the radiography apparatus 7 can acquire a static image satisfying the reference quality information acquired in step S303 (S305). When it is determined that the still image satisfying the reference quality information can be acquired, it is determined that the radiation detector 2 or the radiography apparatus 7 satisfies the capability of imaging of the still image capable of generating the dynamic image.

Then, the controller 201 outputs the result determined in step S305 (S306). For example, the controller 201 causes the display 203 of the dynamic quality control apparatus 6 to display the determination result.

Details of each processing will be described below.

### [2-2. Phantom F]

The phantom F used for imaging in step S301 will be described.

The radiography apparatus 7 images a model used for radiation imaging, which is called a phantom F, instead of imaging using a human body. The phantom F is imaged with settings related to the body thickness, the spatial resolution, the contrast resolution, and the time resolution, but in the present embodiment, the contrast resolution and the spatial resolution are evaluated together. The spatial resolution is evaluated by the size of the object, and the contrast resolution is evaluated by the depth of the object. Here, the body thickness is indicated by the thickness of the plate-shaped portion 401 in the phantom F.

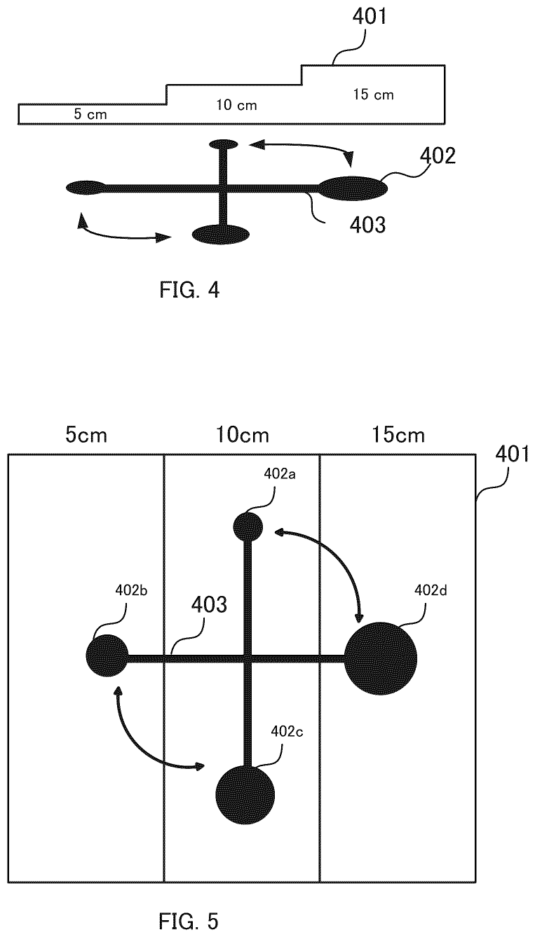

FIG. 4 is a diagram illustrating a configuration of the phantom F. FIG. 5 is a plan view of the phantom F.

For example, the phantom F has a configuration in which a plurality (four in the present embodiment) of granules 402 (spheres in the present embodiment) having different radii are disposed behind the plate-shaped portion 401 and on the ends of a cross-shaped support portion 403, respectively, and the support portion 403 is allowed to rotate around the intersection of the cross. The plate-shaped portion 401 has three plate surface thicknesses of 5 cm, 10 cm, and 15 cm according to thicknesses of muscles, blood volume, and the like at a site to be imaged. The plate-shaped portion 401 is a model part imitating a chest part to a body part of a human body, and the granules 402 are granular model parts of four kinds of sizes for supporting a change in intensity detected by the radiation detector 2 due to a change in blood volume at a diagnostic site. The plate-shaped portion 401 is formed of, for example, an acrylic plate. The granules 402 and the support portion 403 may be made of acrylic resin or another material, similarly to the plate-shaped portion 401. Since the phantom F is a target of radiography, the phantom F may be formed of a material having a certain degree of permeability to the radiation R. At the time of imaging, the phantom F is arranged with the plate surface facing the radiation source 13 such that the thickness direction of the plate-shaped portion 401 is parallel to the irradiation direction of radiation, and is arranged with the rotation axis of the support portion 403 being directed to the radiation detector 2 such that the granules 402 move in a direction intersecting the irradiation direction (thickness direction) when the support portion 403 rotates.

The spatial resolution, the contrast resolution, and the time resolution are measured for each body thickness.

First, the body thickness will be described. For example, when a chest is radiographed, the thickness of the chest of an adult is about 20 cm. Note however that, air does not affect radiation. In the case of imaging a site having little muscle and much air, such as a lung, in the radiography of a human body, the body thickness is small since the part of the body affecting radiation is small. On the other hand, in the case of imaging a site with a large amount of muscle and a small amount of air, such as the heart, the body thickness is large because the body portion affecting radiation is large. The body thickness is determined according to the part to be imaged.

Therefore, the thickness of the plate-shaped portion 401 to be used is set based on the body thickness corresponding to the site to be imaged. The plate-shaped portion 401 of the phantom F used for determination of the evaluation mode in which still images obtained by imaging a lung are used is thin, and is, for example, 5 cm. On the other hand, the plate-shaped portion 401 of the phantom F used for determination of the evaluation mode using the still images of the heart is thick, for example, 10 cm to 15 cm. The thicknesses of the plate-shaped portion 401 are, for example, 5 cm, 10 cm, and 15 cm.

Setting of the phantom F related to the spatial resolution will be described. The spatial resolution is defined by the size (diameter) of a granule of the phantom F in a direction perpendicular to the X-ray irradiation direction. That is, the spatial resolution is the image resolution of a captured image.

The sizes of the granules 402 to be used in the direction perpendicular to the X-ray irradiation direction are set according to the spatial resolution for the determination target. In other words, setting is performed so as to use the granules 402 having a size in the direction perpendicular to the X-ray irradiation direction according to the spatial resolution to be determined. The size of then granule 402 of the phantom F used when the spatial resolution for the determination target is small is small, and the size of the granule 402 of the phantom F used when the spatial resolution as the determination target is large is large. For example, the granules 402 include granules 402a to 402d, and the granule 402a, the granule 402b, the granule 402c, and the granule 402d are used for determination of different spatial resolutions.

Setting of the phantom F related to the contrast resolution will be described. The contrast resolution is defined by the thickness of the phantom F and the size (diameter) of the granules in the X-ray irradiation direction. Detected X-rays are reduced depending on the size (length) of the granule in the X-ray irradiation direction. That is, the contrast resolution is the amount of change in contrast (density) that can be meaningfully distinguished in a captured image. Since the granules of the phantom F in the present embodiment are spherical, the contrast resolution and the spatial resolution are evaluated together.

Assuming that the intensity of the radiation emitted from the radiation source 13 and detected by the radiation detector 2 without any shielding object is 100 and the intensity of the radiation not detected by the radiation detector 2 is 0, the radiography apparatus 7 has a contrast resolution of 1% if it can detect a change in intensity by 1 and a contrast resolution of 10% if it can detect a change in intensity by 10. Furthermore, the intensity detected by the radiation detector 2 changes between a case in which a large amount of blood is contained in a blood vessel and the blood vessel expands and a case in which a small amount of blood is contained and the blood vessel contracts. Whether or not the granule 402 is inserted corresponds to a change in the blood volume of the blood vessel. The intensity detected by the radiation detector 2 changes depending on whether or not the granule 402 is inserted. A change in the intensity detected by the radiation detector 2 is small in a case where the small granule 402 is inserted, and a change in the intensity detected by the radiation detector 2 is large in a case where the large granule 402 is inserted.

That is, the sizes of the granules 402 to be used are set according to contrast resolutions for determination targets. In other words, the granules 402 having sizes corresponding to the contrast resolutions to be determined are set to be used. The size of the granule 402 of the phantom F used when the contrast resolution for the determination target is small (for example, 1%) is small, and the size of the granule 402 of the phantom F used when the contrast resolution as the determination target is large (for example, 10%) is large. For example, the granules 402 include granules 402a to 402d, the granule 402a is a granule used for measurement for 1% contrast resolution, the granule 402b is a granule used for measurement for 3% contrast resolution, the granule 402c is a granule used for measurement for 5% contrast resolution, and the granule 402d is a granule used for measurement for 10% contrast resolution. The granules 402 are, for example, granules corresponding to a contrast of 1% to 10%.

In the present embodiment, the contrast resolution and the spatial resolution are evaluated together. For example, the granule 402a is a granule used for measurement of the spatial resolution of 1 mm, the granule 402b is a granule used for measurement of the spatial resolution of 5 mm, the granule 402c is a granule used for measurement of the spatial resolution of 10 mm, and the granule 402d is a granule used for measurement of the spatial resolution of 50 mm. Note that in the following description, the granules 402a to 402d are referred to as “granules 402” when they are not particularly distinguished from each other.

The setting of the phantom F related to the time resolution will be described.

The time resolution is a frame rate. In a case where only the amplitude of the blood flow needs to be viewed, a frame rate as long as the blood flow cycle is sufficient, and in a case where the waveform of the blood flow needs to be viewed, a frame rate of at least 4 to 8 times the blood flow cycle is required.

When the support portion 403 is rotated, the granules 402 disposed at one ends of the support portion 403 rotate. Along with the rotation of the granules 402, the time when the granules 402 exist in a certain range and the time when the granules 402 do not exist in the certain range periodically change. That is, the intensity detected by the radiation detector 2 periodically changes. When the support portion 403 is rotated quickly, the detected intensity also changes quickly, and when the support portion 403 is rotated slowly, the detected intensity also changes slowly. A frame rate corresponding to the rotational speed of the support portion 403 can be obtained. The support portion 403 can be rotated at, for example, 0.5 to 15 Hz.

Therefore, the rotation speed of the support portion 403 is set according to the time resolution for the determination target.

As described above, the thickness of the plate-shaped portion 401, the sizes of the granules, and the rotation speed of the support part 403 of the phantom F are set according to the body thickness, the contrast resolution, and the time resolution to be determined. The radiography apparatus 7 performs imaging using the set phantom F to generate a still image.

The phantom F has a simple configuration in which the plate-shaped portion 401 have different plate thicknesses, but may have a configuration in which plate-shaped portions having the same thickness but different permeabilities to X-ray are arranged adjacent to each other. Furthermore, instead of the configuration in which the granules 402 having different sizes rotate at a central portion of the plate-shaped portion 401, any configuration may be adopted as long as the granules having different sizes move in respective regions of the plate-shaped portion 401 having different thicknesses. For example, it is possible to adopt a configuration in which granules having different sizes are movably arranged in the respective regions of the plate-shaped portion 401 having different thicknesses, or a configuration in which granules having different sizes linearly reciprocate or circulate across the regions of the plate-shaped portion 401 having different thicknesses. The granules 402 may be spherical, or the sizes (diameters) of the granules 402 in the X-ray irradiation direction may be different from those in the direction perpendicular to the X-ray irradiation direction.

### [2-3. Reference Quality Information]

The reference quality information required for each evaluation mode of S303 will be described. FIG. 8 illustrates the body thickness, the time resolution, and the contrast resolution of the still image necessary for each evaluation mode.

In the present embodiment, determinations for three evaluation modes of the blood flow deficit evaluation mode, the pulmonary artery regurgitation ratio evaluation mode, and the left ventricular ejection capability evaluation mode are exemplified. The blood flow deficit evaluation mode is a mode for generating a dynamic image useful for diagnosing pulmonary embolism. The pulmonary artery regurgitation ratio evaluation mode is a mode for generating a dynamic image useful for diagnosis of Fallot. The left ventricular ejection capability evaluation mode is a mode for generating a dynamic image useful for diagnosing heart failure. Each of the evaluation modes will be described below.

### (Blood Flow Deficit Evaluation Mode)

When the determination of the blood flow deficit evaluation mode for diagnosing pulmonary embolism is performed, it is necessary to grasp the blood flow in the capillaries of the lungs, and therefore it is necessary to image a portion of the lungs. Since the lungs contain a large amount of air, the substantial body thickness is thin. Therefore, since the body thickness necessary for the blood flow deficit evaluation mode may be, for example, 5 cm, the plate-shaped portion 401 used in the phantom F may be thin. The contrast resolution, the spatial resolution, and the time resolution required when X-ray imaging is performed at a body thickness of 5 cm will be described.

Since the change in contrast of the X-ray image due to the change in blood flow in the capillaries is small, it is necessary to have a fine contrast resolution. The contrast resolution required for the blood flow deficit evaluation mode is, for example, 1 to 3%. In addition, since the size of the object (capillaries) is 1 to 5 mm, the spatial resolution required for grasping the blood flow of the capillaries is 1 to 5 mm.

Since it is only necessary to determine the amplitude of the blood flow through the capillaries in the determination of the blood flow deficit evaluation mode, the time resolution needs to be about a frequency of the blood flow cycle. For example, the time resolution required for the blood flow deficit evaluation mode is 0.5 to 2 Hz.

### (Pulmonary Artery Regurgitation Ratio Evaluation Mode)

In a case of performing determination in the pulmonary artery regurgitation ratio evaluation mode in which pulmonary valve regurgitation is diagnosed, it is necessary to grasp the blood flow in the pulmonary artery, and therefore, it is necessary to image a lung portion. Since the lungs contain a large amount of air, the substantial body thickness is thin. Therefore, since the body thickness necessary for the blood flow deficit evaluation mode may be, for example, the 5 cm, the plate-shaped portion 401 used in the phantom F may be thin. The contrast resolution, the spatial resolution, and the time resolution required when X-ray imaging is performed at a body thickness of 5 cm will be described.

Pulmonary valve regurgitation is a state in which backflow occurs at the pulmonary valve and blood leaks back into the right ventricle, causing jet blood flow. Therefore, in diagnosing pulmonary valve regurgitation, it is effective to evaluate the pulmonary artery regurgitation ratio. In order to grasp whether or not the jet blood flow is generated in the pulmonary artery, it is necessary to grasp the blood flow in the pulmonary artery. The pulmonary artery is thicker than the capillary vessel, and a contrast change in radiography due to a blood flow flowing through the pulmonary artery is larger than that of the capillary vessel. That is, a moderate contrast resolution is required as a dynamic image. For example, the required contrast resolution is 3-5%. In addition, since the size of the object (pulmonary arteries) is 5 to 20 mm, the spatial resolution required to express the presence or absence of blood flow in the pulmonary arteries is 5 to 20 mm.

Since it is necessary to grasp the jet blood flow in the pulmonary artery in the determination of the pulmonary artery regurgitation ratio evaluation mode, it is necessary to grasp the waveform. That is, the time resolution requires a frequency about four to eight times the blood flow cycle. For example, the time resolution required for the pulmonary artery regurgitation ratio evaluation mode is the 2.5-10 Hz frequencies.

### (Left Ventricular Ejection Capability Evaluation Mode)

Since heart failure is classified according to the left ventricular ejection fraction (LVEF), it is effective to evaluate the left ventricular ejection capability in diagnosing heart failure, and thus it is necessary to image the heart. Since the heart is less permeable to radiation than the lungs, the body thickness is relatively large, and thus the body thickness required for the left ventricular ejection capability evaluation mode is large. For example, the necessary body thickness is 10-15 cm, and the plate-shaped portion 401 used in the phantom F is thick. The contrast resolution, spatial resolution, and time resolution required when X-ray imaging is performed at a body thickness of 10-15 cm will be described.

Since the blood flow rate in the ventricle is large, a change in contrast of radiography due to a change in blood flow rate in the ventricle is large. That is, as a dynamic image, the contrast resolution does not need to be so high. For example, the required contrast resolution is 5-10%. In addition, since the size of the object (cardiac chamber) is 10 to 50 mm, the spatial resolution required to express the presence or absence of blood flow in the heart is 10 to 50 mm.

In the determination of the left ventricular ejection capability evaluation mode, the amplitude of the blood flow sent out from the left ventricle according to the heartbeat may be determined, and therefore, a frequency corresponding to the blood flow is necessary. That is, the time resolution needs to be a frequency corresponding to the blood flow. For example, the time resolution required for determination of the left ventricular ejection capability evaluation mode is a frequency of 1 to 4 Hz.

### [2-4. Determination of Quality of Still Image]

Here, details of step S304 will be described. Step S304 is a step of determining whether or not the still image of the phantom F captured by the radiography apparatus 7 satisfies the reference quality.

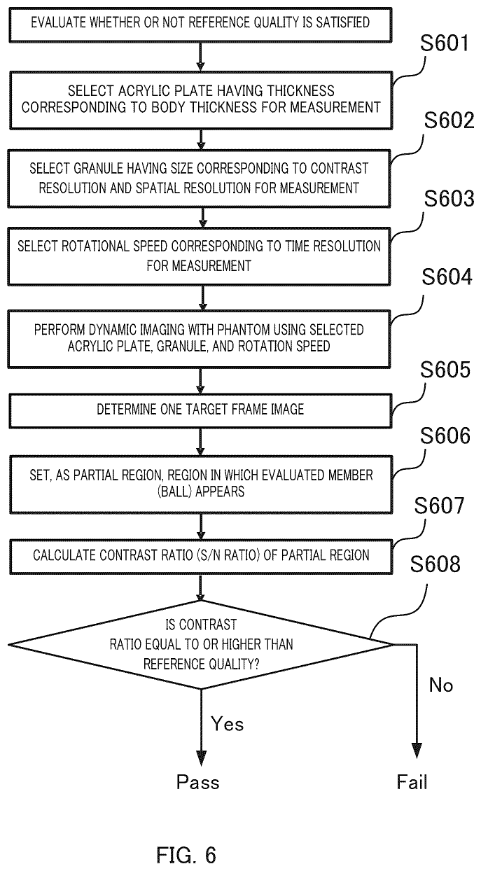

FIG. 6 illustrates details of determining whether or not a still image satisfies the reference quality.

The controller 201 receives an input by a user operation of the operation unit 205 of the dynamic quality control apparatus 6 (hereinafter, simply referred to as “operation unit 205”) and sets the thicknesses of the plate-shaped portion 401 corresponding to the body thicknesses of the determination target (S601). In addition, the controller 201 receives an input by a user operation of the operation unit 205 and sets the granule 402 (any one of granules 402a, 402b, 402c, and 402d) having a size corresponding to the contrast resolution and the spatial resolution for the determination target (S602). Further, the controller 201 receives an input by a user operation of the operation unit 205 and sets the rotation speed of the support portion 403 corresponding to the time resolution for the determination target (S603).

Then, the radiography apparatus 7 performs dynamic imaging using the phantom F (S604). That is, the radiography apparatus 7 irradiates the phantom F with radiation by pulse irradiation or continuous irradiation, and obtains a dynamic image including a plurality of still images in which the phantom F is the subject S. Thus, the controller 201 acquires the plurality of still images that have been captured. Note that during this time, the granules 402 of the phantom F rotationally move at a set rotational speed in accordance with the rotational movement of the support portion 403 at the set rotational speed. By connecting the rotation center (intersecting portion of the cross) of the support portion 403 to an output shaft of a motor (not illustrated), the support portion 403 and the granules 402 can be rotated by driving the motor. In addition, the rotation speed of the support portion 403 can be switched by switching the rotation speed of the motor.

In addition, in a case of measuring an object whose speed smoothly changes, acceleration (angular acceleration) may be changed in addition to the rotation speed.

Then, the controller 201 determines one target static image from a plurality of static images obtained by the dynamic imaging (S605).

The controller 201 receives an input by a user operation of the operation unit 205, and selects a region in which the granule 402 appears from the determined still image (S606). When the phantom F in the plan view illustrated in FIG. 5 is used, four patterns of combinations of the thickness of the plate-shaped portion 401 and the size of the granule 402 are photographed in one still image. For example, in FIG. 5, the contrast resolution of 3% and the body thickness of 5 cm (pattern 1), the contrast resolution of 1% and the body thickness of 10 cm (pattern 2), the contrast resolution of 5% and the body thickness of 10 cm (pattern 3), and the contrast resolution of 10% and the body thickness of 15 cm (pattern 4) can be measured by one imaging. When the support portion 403 is rotated, a still image in which the combinations of the thicknesses of the plate-shaped portion 401 and the granules 402 are changed with time is captured. Therefore, a still image in which a desired combination of the thicknesses of the plate-shaped portion 401 and the granules 402 is captured may be determined from a plurality of captured still images, and a region in which the desired combination appears may be selected from the determined still image. This selection may be manually performed by the user based on a visual observation result, or may be automatically performed by the controller 201 in accordance with a predetermined criterion.

Then, the controller 201 calculates the contrast ratio of the region in which the granule appears to the region in which the granule does not appear (S607).

Next, the controller 201 determines whether or not the calculated contrast ratio satisfies the reference quality (S608). The controller 201 outputs “Pass” when the contrast ratio is equal to or higher than the reference value (YES in S608), and outputs “Fail” when the contrast ratio is lower than the reference value (NO in S608). Note that the reference quality is, for example, S/N (signal-to-noise ratio)=1.

If the result is “Pass”, it is determined that the radiography apparatus 7 has the capability to perform imaging with the body thickness, the contrast resolution, the spatial resolution, and the time resolution that correspond to the setting values of the phantom F acquired in steps S601 to S603 (S302).

For example, in a case where an image has been captured using the phantom F in which the support portion 403 has been rotated at 2 Hz while using a granule having a size corresponding to the thickness of the plate-shaped portion 401 corresponding to a body thickness of 5 cm and a contrast resolution of 10%, the body thickness of 5 cm, the contrast resolution of 10%, and the time resolution of 2 Hz are determined to be “Pass” if the contrast ratio between the “region in which the granule appears” and the “region in which no granule appears” in the captured still image is higher than or equal to the reference quality.

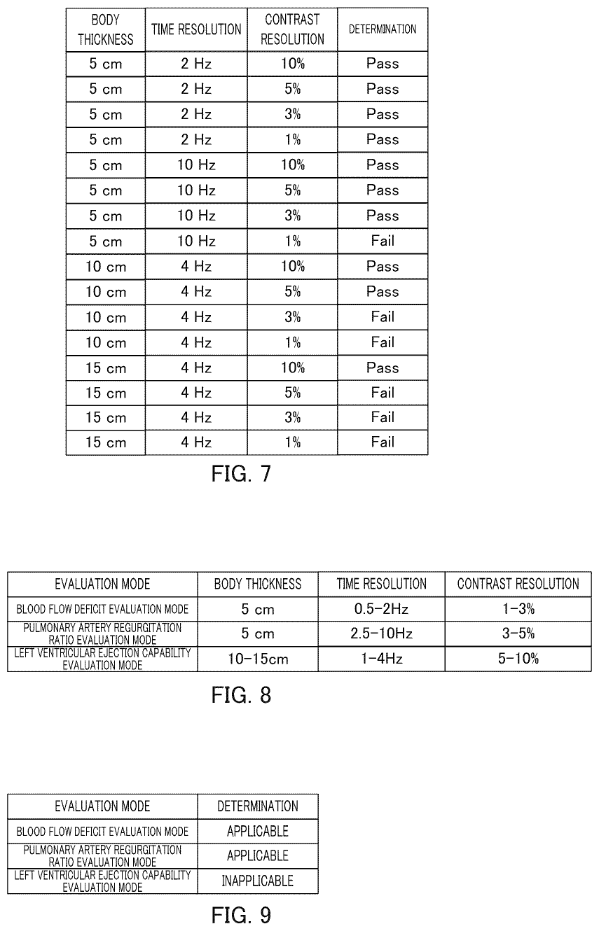

FIG. 7 illustrates an example of the result of the determination in step S608 (S304). FIG. 7 illustrates a result of imaging performed by the radiography apparatus with 16 different settings of phantom F. Since the spatial resolution is evaluated together with the contrast resolution, the spatial resolution is not illustrated in FIG. 7. When the phantom F in the plan view illustrated in FIG. 5 is used, still images of four combinations of the thicknesses of the plate-shaped portion 401 and the sizes of the granules are captured in one still image, and an arbitrary combination of the body thickness and the size of the granule can be obtained by selection of the still image and selection of the region, so that 16 results illustrated in FIG. 7 can be obtained by imaging performed with four set rotation speeds of the support portion 403.

### [2-5. Determination of Each Evaluation Mode]

In step S305, it is determined whether or not the radiography apparatus 7 has the body thickness, contrast resolution, and time resolution capability acquired in step S303 and necessary for each evaluation mode.

FIG. 8 illustrates the resolution acquired in step S303 and required for performing dynamic state processing in each evaluation mode. Since the spatial resolution is evaluated together with the contrast resolution, the spatial resolution is not illustrated in FIG. 8.

For example, when the body thickness is 5 cm in the blood flow deficit evaluation mode, the required time resolution is 0.5 to 2 Hz, and the required contrast resolution is 1 to 3%.

If at least one of the still images used in the evaluation mode does not satisfy the quality, an accurate dynamic image is not output. Therefore, if all of the still images captured with the settings combining the resolutions necessary for the evaluation mode are “Pass”, the evaluation mode is determined to be “Pass (or Applicable)”, and if even one of the still images is determined to be “Fail”, the evaluation mode is determined to be “Fail (or Inapplicable)”.

In the blood flow deficit evaluation mode, the time resolution is 0.5 to 2 Hz and the contrast resolution is 1 to 3% at the time of the body thickness of 5 cm, and therefore, the blood flow deficit evaluation mode is determined to be “Pass” in a case where all the determinations with “body thickness of 5 cm, time resolution of 0.5 Hz, contrast resolution of 1%”, “body thickness of 5 cm, time resolution of 0.5 Hz, contrast resolution of 3%”, “body thickness of 5 cm, time resolution of 2 Hz, contrast resolution of 1%”, and “body thickness of 5 cm, time resolution of 2 Hz, contrast resolution of 3%” are “Pass”.

According to the generated information on the body thickness, the contrast resolution, and the time resolution illustrated in FIG. 7, the determination result for measurement performed with a body thickness of 5 cm, a rotation speed of 2 Hz, and a contrast resolution of 1% is “Pass”. In addition, the determination result for the measurement performed with the body thickness of 5 cm, the rotation speed of 2 Hz, and the contrast resolution of 3% is also “Pass”. The determination results for “body thickness of 5 cm, time resolution of 0.5 Hz, contrast resolution of 1%” and “body thickness of 5 cm, time resolution of 0.5 Hz, contrast resolution of 3%” are not present. New imaging may be performed using the phantom F. However, since it is clear that the time resolution of 0.5 Hz may be lower than the time resolution of 2 Hz, it can be estimated that the time resolution of 0.5 Hz is also “Pass” if the time resolution of 2 5 Hz is “Pass”. That is, the dynamic quality control apparatus may estimate the time resolution of 0.5 Hz as “Pass” without newly setting the phantom F and performing the measurement.

Therefore, since the quality of all the still images necessary for the evaluation mode is “Pass”, the blood flow deficit evaluation mode is determined to be “Pass” (“Applicable” in FIG. 9).

Similarly, the pulmonary artery regurgitation ratio evaluation mode is determined to be “Pass” (“Applicable” in FIG. 9).

On the other hand, regarding the left ventricular ejection capability evaluation mode, since “body thickness of 15 cm, time resolution of 4 Hz, and contrast resolution of 5%” are “Fail”, the left ventricular ejection capability evaluation mode is determined to be “Fail” (“Inapplicable” in FIG. 9).

### [2-6. Conclusion]

By referring to the output determination result, it is possible to determine whether or not the radiography apparatus 7 that has performed imaging has the imaging capability in each evaluation mode.

For example, when the determination result of FIG. 9 is output, the radiography apparatus 7 that has obtained the result of FIG. 7 has the imaging capability in the case of performing the blood flow deficit evaluation mode and the pulmonary artery regurgitation ratio evaluation mode, but does not have the imaging capability in the case of performing the left ventricular ejection capability evaluation mode. That is, when diagnosing heart failure, an effective dynamic image cannot be obtained even if the radiography apparatus 7 is used. However, the radiography apparatus 7 can obtain a dynamic image effective for use in diagnosis of pulmonary embolism and pulmonary valve regurgitation.

Therefore, since it is possible to determine whether or not the radiography apparatus 7 can obtain a still image having a quality necessary for each evaluation mode, that is, whether or not the radiography apparatus 7 has a capability necessary for performing the evaluation mode, it is possible to control the quality of dynamic imaging.

The radiography apparatus 7 acquires the reference quality information by using the phantom F and it is determined whether or not the reference quality necessary for each evaluation mode is obtained. It is thus possible to determine whether or not the radiography apparatus 7 can obtain a dynamic image necessary for each evaluation mode.

It becomes more and more difficult for the quality reference information of the dynamic image to satisfy the reference quality as the body thickness increases, the time resolution (frequency) increases, and the contrast resolution (granule) decreases. Therefore, the propriety of the analysis for each evaluation mode may be determined using the quality reference information of the dynamic image having the largest numerical value of the body thickness, the lowest contrast resolution, and the highest time resolution.

### Modification Example

In the phantom F, four granules 402 having different radii are arranged at the end portions of the cross-shaped support portion 403, but the number of the granules 402 arranged at the end portions of the support portion 403 may be other than four. Further, the support portion 403 may not be cross-shaped. The granules 402 having different radii may be changeably disposed at one end portion of the support portion 403. The granules 402 having the same radius may be disposed at the end portions of the support portion 403, and the granules 402 may be disposed in a changeable manner. Other phantoms may also be used. For example, a CDRAD phantom may be fixed to an acrylic plate in a rotatable manner. Instead of changing the thickness of the plate-shaped portion 401 in accordance with the body thickness, the radiolucency may be changed by changing the material or the like of the plate-shaped portion 401.

The reference quality information exemplifies the quality corresponding to the three evaluation modes, but the number of evaluation modes is not limited to three. Quality corresponding to another evaluation mode may be set. The reference quality information is set for each evaluation mode, but a plurality of qualities such as quality of A-rank allowing a high-definition dynamic image to be displayed and quality of B-rank allowing a normal dynamic image to be displayed may be set for each evaluation mode.

The determination in step S305 may indicate a quality level of the provided dynamic image instead of a comparison result between the imaging capability of the radiation detector 2 or the radiography apparatus 7 and the required imaging capability, such as whether the determination is possible or not. For example, as the quality of the dynamic image provided in the evaluation mode using the captured still image, a ratio to the highest quality level, for example, a quality level of 80%, a quality level of 60%, or the like may be determined and output. As another example, the quality level of the provided dynamic image and the reference quality level may be displayed side by side.

If there are a plurality of radiography apparatuses, it is possible to determine in advance which radiography apparatus can perform dynamic imaging necessary for diagnosis, and reserve a radiography apparatus necessary for diagnosis.

Although the embodiments have been described above with reference to the drawings, the present disclosure is not limited to such examples. It is obvious that a person skilled in the art can conceive of various change examples or modification examples within the scope described in the claims It is to be understood that such changes or modifications also belong to the technical scope of the present disclosure. Furthermore, the constituent elements in the embodiments may be combined as appropriate without departing from the spirit of the present disclosure.

(1) A capability determination method performed by a dynamic quality control apparatus according to an embodiment of the present disclosure includes: acquiring an imaging capability from an image obtained by a radiation detector by irradiation of a model with radiation; and outputting the acquired imaging capability in such a manner as to indicate a relation with a required imaging capability for a predetermined dynamic analysis.

(2) The capability determination method according to an embodiment of the present disclosure is the capability determination method (1), in which the outputting includes outputting a comparison result between the acquired imaging capability and the required imaging capability for the predetermined dynamic analysis.

(3) The capability determination method according to an embodiment of the present disclosure is the capability determination method of (1), in which the required imaging capability for the predetermined dynamic analysis is an imaging capability for each dynamic analysis, and the outputting includes outputting whether or not the radiation detector has the imaging capability for each dynamic analysis.

(4) The capability determination method according to an embodiment of the present disclosure is the capability determination method of (1), in which the imaging capability is an imaging capability of the radiation detector.

(5) The capability determination method according to an embodiment of the present disclosure is the capability determination method of (1), in which the imaging capability is an imaging capability of a radiography apparatus.

(6) The capability determination method according to an embodiment of the present disclosure is the capability determination method of (5), in which the radiography apparatus includes a radiation generating apparatus and the radiation detector.

(7) The capability determination method according to an embodiment of the present disclosure is the capability determination method of (6), in which the radiography apparatus further includes a console.

(8) The capability determination method according to an embodiment of the present disclosure is the capability determination method of (1), in which the model includes a plurality of regions having different radiolucencies.

(9) The capability determination method according to an embodiment of the present disclosure is the capability determination method of (1), in which the model includes a plurality of granules having different sizes.

(10) The capability determination method according to an embodiment of the present disclosure is the capability determination method of (1), in which the imaging capability includes a contrast resolution that is an amount of change in contrast of a captured image.

(11) The capability determination method according to an embodiment of the present disclosure is the capability determination method according to (1), in which the imaging capability includes a time resolution that is a frame rate.

(12) The capability determination method according to an embodiment of the present disclosure is the capability determination method of (1), in which the imaging capability includes a spatial resolution that is an image resolution of a captured image.

(13) The capability determination method according to an embodiment of the present disclosure is the capability determination method of (1), in which the predetermined dynamic analysis is a dynamic analysis relevant to pulmonary embolism, and the required imaging capability includes a contrast resolution of 1 to 3%, a spatial resolution of 1 to 5 mm, and a frame rate of 0.5 to 2.0 Hz when a body thickness is 5 cm.

(14) The capability determination method according to an embodiment of the present disclosure is the capability determination method of (1), in which the predetermined dynamic analysis is a dynamic analysis relevant to pulmonary valve regurgitation, and the required imaging capability includes a contrast resolution of 3 to 5%, a spatial resolution of 5 to 20 mm, and a frame rate of 2.5 to 10 Hz when a body thickness is 5 cm.

(15) The capability determination method according to an embodiment of the present disclosure is the capability determination method of (1), in which the predetermined dynamic analysis is a dynamic analysis relevant to heart failure, and the required imaging capability includes a contrast resolution of 5 to 10%, a spatial resolution of 10 to 50 mm, and a frame rate of 1.0 to 4 Hz when a body thickness is 10 to 15 cm.

(16) A capability determination apparatus according to an embodiment of the present disclosure includes: an acquirer that acquires an imaging capability from an image obtained by a radiation detector by irradiation of a model with radiation; and an output that outputs the acquired imaging capability in such a manner as to indicate a relation with a required imaging capability for a predetermined dynamic analysis.

(17) A capability determination program according to an embodiment of the present disclosure causes a computer to execute the capability determination method of (1).

(18) A phantom according to an embodiment of the present disclosure is a phantom used as a model in the capability determination method of (1), and the phantom includes a plurality of regions having different radiolucencies and a plurality of granules having different sizes, the plurality of granules being disposed at positions downstream of the plurality of regions along passing radiation.

(19) The phantom according to an embodiment of the present disclosure is the phantom of (18), in which the plurality of granules having different sizes are movable.

(20) A determination method for determining a dynamic imaging capability of a radiography apparatus according to an embodiment of the present disclosure is a determination method for determining a dynamic imaging capability of a radiography apparatus and includes: preparing a model whose radiolucency and movement speed are changeable; acquiring a plurality of images continuously captured by irradiation of the model with radiation, while selecting a combination of the radiolucency and the movement speed of the model; comparing a resolution of each the plurality of image acquired and a reference value set for each imaging purpose; and determining, based on a result of the comparison, whether or not the radiography apparatus has a required imaging capability.

(21) The determination method for determining a dynamic imaging capability of a radiography apparatus according to an embodiment of the present disclosure is the determination method according to (20), in which the resolution of the image is a contrast resolution, a spatial resolution, or a time resolution.

(22) A determination apparatus for determining a dynamic imaging capability of a radiography apparatus according to an embodiment of the present disclosure is a determination apparatus for determining a dynamic imaging capability of a radiography apparatus and includes: an image acquirer that acquires a plurality of images continuously captured by irradiation of the model with radiation, while selecting a combination of a radiolucency and a movement speed of a model whose radiolucency and movement speed are changeable; a comparison section that compares a resolution of each the plurality of image acquired and a reference value set for each imaging purpose; and a determiner that determines, based on a result of the comparison, whether or not the radiography apparatus has a required imaging capability.

(23) The determination apparatus for determining a dynamic imaging capability of a radiography apparatus according to an embodiment of the present disclosure is the determination apparatus according to (22), in which the resolution of the image is a contrast resolution, a spatial resolution, or a time resolution.

### Industrial Applicability

The present disclosure is useful for quality control in dynamic imaging using radiation.

Although embodiments of the present invention have been described and illustrated in detail, the disclosed embodiments are made for purpose of illustration and example only and not limitation. The scope of the present invention should be interpreted by terms of the appended claims

What is claimed is:

1. A capability determination method performed by a dynamic quality control apparatus, the capability determination method comprising:
acquiring an imaging capability from an image obtained by a radiation detector by irradiation of a model with radiation; and
outputting the acquired imaging capability in such a manner as to indicate a relation with a required imaging capability for a predetermined dynamic analysis.

2. The capability determination method according to claim 1, wherein
the outputting includes outputting a comparison result between the acquired imaging capability and the required imaging capability for the predetermined dynamic analysis.

3. The capability determination method according to claim 1, wherein:
the required imaging capability for the predetermined dynamic analysis is an imaging capability for each dynamic analysis, and
the outputting includes outputting whether or not the imaging capability for each dynamic analysis is included.

4. The capability determination method according to claim 1, wherein
the imaging capability is an imaging capability of the radiation detector.

5. The capability determination method according to claim 1, wherein
the imaging capability is an imaging capability of a radiography apparatus.

6. The capability determination method according to claim 5, wherein
the radiography apparatus includes a radiation generating apparatus and the radiation detector.

7. The capability determination method according to claim 6, wherein
the radiography apparatus further includes a console.

8. The capability determination method according to claim 1, wherein
the model includes a plurality of regions having different radiolucencies.

9. The capability determination method according to claim 1, wherein
the model includes a plurality of granules having different sizes.

10. The capability determination method according to claim 1, wherein
the imaging capability includes a contrast resolution that is an amount of change in contrast of a captured image.

11. The capability determination method according to claim 1, wherein
the imaging capability includes a time resolution that is a frame rate.

12. The capability determination method according to claim 1, wherein
the imaging capability includes a spatial resolution that is an image resolution of a captured image.

13. The capability determination method according to claim 1, wherein:
the predetermined dynamic analysis is a dynamic analysis relevant to pulmonary embolism, and
the required imaging capability includes a contrast resolution of 1 to 3%, a spatial resolution of 1 to 5 mm, and a frame rate of 0.5 to 2.0 Hz when a body thickness is 5 cm.

14. The capability determination method according to claim 1, wherein:
the predetermined dynamic analysis is a dynamic analysis relevant to pulmonary valve regurgitation, and
the required imaging capability includes a contrast resolution of 3 to 5%, a spatial resolution of 5 to 20 mm, and a frame rate of 2.5 to 10 Hz when a body thickness is 5 cm.

15. The capability determination method according to claim 1, wherein:
the predetermined dynamic analysis is a dynamic analysis relevant to heart failure, and
the required imaging capability includes a contrast resolution of 5 to 10%, a spatial resolution of 10 to 50 mm, and a frame rate of 1.0 to 4 Hz when a body thickness is 10 to 15 cm.

16. A capability determination apparatus, comprising:
an acquirer that acquires an imaging capability from an image obtained by a radiation detector by irradiation of a model with radiation; and
an output that outputs the acquired imaging capability in such a manner as to indicate a relation with a required imaging capability for a predetermined dynamic analysis.

17. A computer-readable recording medium storing a capability determination program for causing a computer to execute the capability determination method according to claim 1.

18. A phantom used as a model in the capability determination method according to claim 1, the phantom comprising:
a plurality of regions having different radiolucencies and a plurality of granules having different sizes, the plurality of granules being disposed at positions downstream of the plurality of regions along passing radiation.

19. The phantom according to claim 18, wherein
the plurality of granules having different sizes are movable.

20. A determination method for determining a dynamic imaging capability of a radiography apparatus, the determination method comprising:
preparing a model whose radiolucency and movement speed are changeable;
acquiring a plurality of images continuously captured by irradiation of the model with radiation, while selecting a combination of the radiolucency and the movement speed of the model;
comparing a resolution of each the plurality of image acquired and a reference value set for each imaging purpose; and
determining, based on a result of the comparison, whether or not the radiography apparatus has a required imaging capability.

21. The determination method for determining a dynamic imaging capability of a radiography apparatus according to claim 20, wherein
the resolution of the image is a contrast resolution, a spatial resolution, or a time resolution.

22. A determination apparatus for determining a dynamic imaging capability of a radiography apparatus, comprising:
a hardware processor, wherein the hardware processor
acquires a plurality of images continuously captured by irradiation of a model with radiation, while selecting a combination of a radiolucency and a movement speed of the model whose radiolucency and movement speed are changeable;
compares a resolution of each the plurality of image acquired and a reference value set for each imaging purpose; and
determines, based on a result of the comparison, whether or not the radiography apparatus has a required imaging capability.

23. The determination apparatus for determining a dynamic imaging capability of a radiography apparatus according to claim 22, wherein
the resolution of the image is a contrast resolution, a spatial resolution, or a time resolution.
